(19) United States
(12) Patent Application Publication
(10) Pub. No.: US 2009/0124980 A1
(43) Pub. Date: May 14, 2009

(54) SYRINGE

(75) Inventor: Xue Li Chen, Weihai (CN)

Correspondence Address: HOWSON & HOWSON LLP, 501 OFFICE CENTER DRIVE, SUITE 210, FORT WASHINGTON, PA, 19034, US

(73) Assignee: SHANDONG WEIGAO GROUP MEDICAL POLYMER CO., LTD., Weihai (CN)

(21) Appl. No.: 12/170,554

(22) Filed: Jul. 10, 2008

Related U.S. Application Data
(60) Provisional application No. 60/959,020, filed on Jul. 10, 2007.

(51) Int. Cl. A61M 5/32

(52) U.S. Cl. 604/196

(57) ABSTRACT

In a safety syringe in which a needle and needle holder can be withdrawn into a barrel after use, the needle holder is provided with an insert having forwardly and inwardly extending resilient fingers that are cammed outward by an enlarged head formed on a projection of the plunger of the syringe, and engage a proximally facing surface on the head to connect the needle holder positively to the plunger so that the needle holder can be withdrawn into the barrel of the syringe.

### CROSS-REFERENCE TO RELATED APPLICATION

This application claims priority on the basis of U.S. provisional patent application Ser. No. 60/959,020, filed Jul. 10, 2007.

### FIELD OF THE INVENTION

This invention relates to safety syringes, and more particularly to disposable safety syringes of the type in which, following a single use, for example the administration of an injection, the needle of the syringe can be engaged by the plunger, and withdrawn to a safe position within the interior of the syringe barrel, where it cannot cause injury.

### BACKGROUND OF THE INVENTION

In a typical safety syringe of the above-mentioned type, a needle holder is held, either by friction, or by a releasable positive locking mechanism, at a distal end of a syringe barrel. Safety syringes of this type are described in numerous United States patents, including the following: U.S. Pat. No. 4,927,414 May 22, 1990, U.S. Pat. No. 4,950,241, Aug. 21, 1990, U.S. Pat. No. 4,995,870 Feb. 26, 1991, U.S. Pat. No. 5,125,899 Jun. 30, 1992, U.S. Pat. No. 5,290,233 Mar. 1, 1994, U.S. Pat. No. 5,328,475 Jul. 12, 1994, U.S. Pat. No. 5,338,304 Aug. 16, 1994, U.S. Pat. No. 5,431,631 Jul. 11, 1995, U.S. Pat. No. 5,431,632 Jul. 11, 1995, U.S. Pat. No. 5,533,975 Jul. 9, 1996, U.S. Pat. No. 5,569,203 Oct. 29, 1996, U.S. Pat. No. 5,575,774 Nov. 19, 1996, U.S. Pat. No. 5,578,015 Nov. 26, 1996, U.S. Pat. No. 5,772,687 Jun. 30, 1998, U.S. Pat. No. 5,968,020 Oct. 19, 1999, U.S. Pat. No. 5,993,419 Nov. 30, 1999, U.S. Pat. No. 6,033,385 Mar. 7, 2000, U.S. Pat. No. 6,468,246 Oct. 22, 2002, U.S. Pat. No. 6,488,657 Dec. 3, 2002 and U.S. Pat. No. 6,821,266 Nov. 23, 2004.

A needle is connected to the needle holder by a “Luer” fitting, that is, a fitting in which a hub in which the needle is fixed is engaged with threads provided on the needle holder. Thus, the needle can be readily engaged with the needle holder by grasping its hub, engaging the threads, and rotating the hub relative to the needle holder.

The plunger of the syringe has a stopper that cooperates with the inside of the barrel to force liquid out of the barrel through the needle. The plunger and the needle holder have cooperable coupling elements, forming a latch by which the needle holder becomes automatically connected to the plunger when the plunger is pushed to its extreme distal position. When the plunger and the needle holder are connected by the latch, the needle holder, with the needle attached to it, can be withdrawn into the barrel by pulling the plunger.

A problem with conventional safety syringes is that in many cases, the medication to be injected is expensive, and consequently it is desirable to avoid waste by minimizing the volume of medication left in the syringe following completion of an injection. The space containing the remainder of the medication is sometimes referred to as “dead space.” In a safety syringe the minimization of dead space is made more difficult by the need for coupling elements.

In some safety syringes, a latching mechanism is provided to lock the needle holder positively to the distal end of the barrel. The latching mechanism is released only when engaged by the plunger. Examples of such mechanisms are described in U.S. Pat. Nos. 6,468,246 and 6,488,657. In these patents some precautions are taken to minimize dead space.

Some simpler safety syringes, such as the one described in U.S. Pat. No. 5,993,419, utilize friction, or cooperating resilient members, to keep the needle holder in place. In these syringes, coupling members on the needle holder and the plunger engage each other so that, when the plunger is withdrawn, the frictional or resilient force which keeps the needle holder in place is overcome. In the safety syringe of U.S. Pat. No. 5,993,419, there is a dead space. The dead space can be reduced in a safety syringe in which the needle holder is kept in place by friction or resilient force, but various mechanical problems are encountered in the design of such syringes. One problem is that the frictional or resilient force keeping the needle holder in place in the barrel must be relatively high. The coupling mechanism by which the plunger is connected to the needle holder must be sufficient to withstand the relatively large force needed to disengage the needle holder from the end of the barrel. However, it can be difficult to engage coupling elements that are capable of withstanding a relatively large pulling force. In a safety syringe, it is very desirable for the coupling elements to be easily and reliably engaged. Moreover, it is important to minimize the dead space. These requirements are not easily satisfied in conventional safety syringe design.

Accordingly, there is a need for a simple and reliable safety syringe in which the needle holder is normally held in place by friction or by a resilient force, in which the needle holder can be easily and reliably engaged by the plunger upon completion of the administration of an injection, and in which dead space is minimized.

### SUMMARY OF THE INVENTION

In accordance with the invention, the above-mentioned problems are addressed by incorporation of a specially designed insert into the needle holder. The insert has unitary, forwardly and inwardly protruding fingers, disposed in windows in the insert. The fingers are spread outward by the head of a protrusion on the plunger, and their tips positively engage a proximally facing surface on the head of the protrusion, enabling the needle holder and needle to be withdrawn into the barrel reliably by pulling the plunger.

More specifically, the syringe in accordance with the invention comprises a barrel, having a cylindrical inner wall. The barrel is elongated along a longitudinal barrel axis and has a proximal end and a distal end, an opening at the proximal end, and a hollow extension at the distal end. A stopper, in fluid-tight sliding engagement with the cylindrical inner wall of the barrel, is connected to a plunger, which extends outward from the barrel through the opening at the proximal end. A needle holder is fitted in the hollow extension of the barrel, and has a passage extending from the proximal end to the distal end of the needle holder.

When the plunger is moved distally beyond a predetermined limit, coupling elements on the needle holder and the plunger are engageable with each other for coupling the plunger to the needle holder and thereby enabling the needle holder, and a needle hub and needle connected to the needle holder, to be withdrawn into the interior of the barrel by the exertion of a proximal pulling force on the plunger.

Mutually engaged retaining elements on the needle holder and in the hollow extension, resist proximal movement of the needle holder but permit proximal movement of the needle holder upon application of a proximal pulling force on the plunger when the coupling elements on the needle holder and the plunger are engaged.

The improvement resides in the particular features of the coupling elements, especially in the use of an insert having resilient fingers.

Specifically, the coupling element on the needle holder comprises an insert fitted into the passage in the needle holder and restrained against proximal movement relative to the needle holder. The insert has a base at its proximal end, and a side wall at least partially surrounding, and defining, an axial passage through the insert. The side wall is interrupted by a plurality of windows, and a resilient finger, disposed in each of the windows, extends distally from the base. Each finger is unitary with the base of the insert and has a tip remote from the base. When the fingers are relaxed, the tip of each finger is located at least partly within the axial passage of the insert.

The coupling element on the plunger comprises an axial protrusion having a head with a proximally facing surface. The size of the head is such that the head is capable of passing through the axial passage of the insert, but sufficient to engage the fingers of the insert and spread the tips of the fingers of the insert radially outward as the axial protrusion is moved distally.

When the plunger is moved distally by a distance such that the proximally facing surface of the head of the axial protrusion passes the tips of the fingers of the insert, the tips of the fingers are engageable with the proximally facing surface of the head. The engagement of the proximally facing surface with the tips of the fingers enables the needle holder, and a needle connected to the needle holder, to be withdrawn into the barrel as the plunger is pulled in the proximal direction.

The syringe is preferably used with a hollow needle fixed to a needle hub. The needle hub is removably engageable with the needle holder to establish a rigid connection of the needle and needle hub to the needle holder and to establish a fluid-conducting connection of the hollow needle to the passage of the needle holder.

The mutually engaged retaining elements on the needle holder, and in the hollow extension, preferably require the magnitude of a proximal pulling force exerted on the plunger to exceed a predetermined magnitude in order to permit proximal movement of the needle holder. Retaining elements of the frictional or resilient type operate in this manner.

### BRIEF DESCRIPTION OF THE DRAWINGS

FIG. 1 is a longitudinal section of a syringe according to a preferred embodiment of the invention;

FIG. 2 is a side elevation, partly in section, of the needle holder in the syringe of FIG. 1;

FIG. 3 is a side elevation, partly in section, of a needle holder insert which cooperates with a projection on the plunger to connect the plunger to the needle holder when the plunger is fully depressed;

FIG. 4 is a bottom plan view of the needle holder insert of FIG. 3;

FIG. 5 is a partially broken-away, enlarged, longitudinal section of the distal end of the syringe of FIG. 1, showing the plunger about to engage the needle holder; and

FIG. 6 is an enlarged longitudinal section of the distal end of the syringe, showing the plunger engaged with the needle holder.

### DESCRIPTION OF THE PREFERRED EMBODIMENT

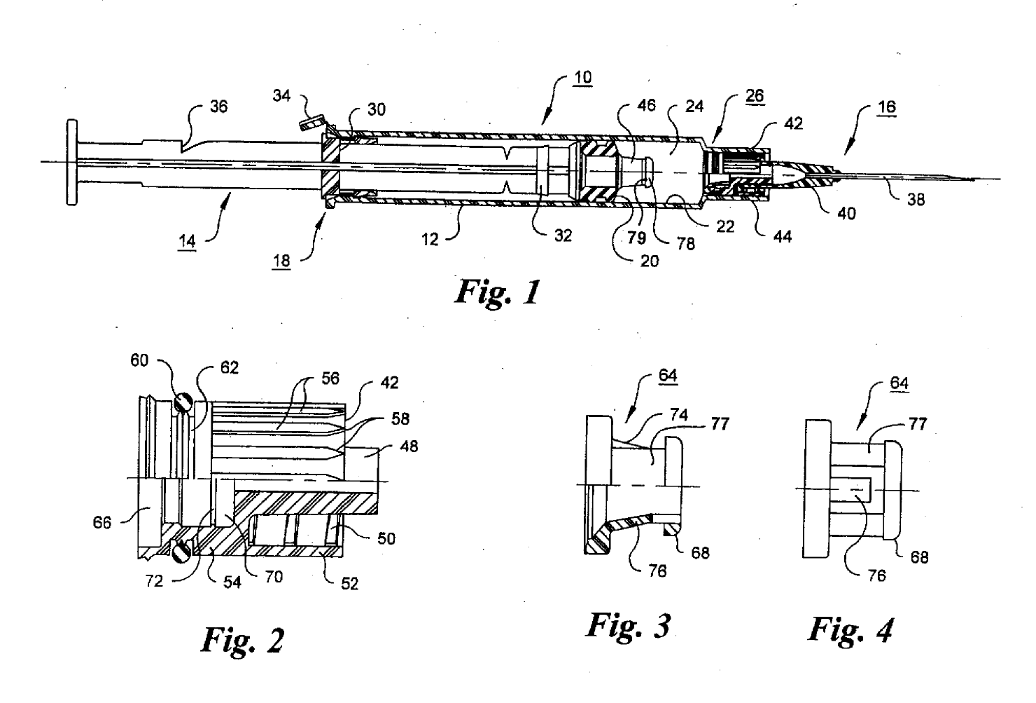

As shown in FIG. 1, the syringe 10 in accordance with the invention comprises a barrel 12, a plunger 14, and a needle assembly 16. The plunger extends into the barrel through an opening at the proximal end 18 of the barrel, and is provided with an elastomeric stopper 20, which is in fluid-tight, sliding engagement with a cylindrical inner wall 22 of the barrel to define a fluid chamber 24, which extends from the stopper to the distal end 26 of the barrel.

The plunger has a cross-shaped transverse cross-section, and slides through an insert 30 fitted to the proximal end of the barrel. The insert cooperates with an annulus 32 on the plunger to lock the plunger in a retracted condition when the plunger is fully withdrawn after use. The insert 30, and the manner in which it cooperates with annulus 32, are described in more detail in U.S. Pat. No. 6,821,266, the specification of which is incorporated by reference.

A hinged tab 34 is provided at the proximal end of the barrel. The syringe is supplied to the user with this hinged tab in engagement with a notch 36 in the plunger to prevent the plunger from being pushed forward inadvertently before use. The tab and notch are also described in U.S. Pat. No. 6,821,266.

The needle assembly 16, which consists of a hollow needle 38 fitted to a needle hub 40, is removably connected to a needle holder 42 located within a hollow extension 44 formed at the distal end 26 of the barrel. A projection 46 on the distal end of the plunger can enter the needle holder when the plunger is pushed forward to the limit of its travel.

The needle holder 42, which is shown in greater detail in FIG. 2, is a molded resin unit having a tubular projection 48 adapted to fit into the needle hub 40 (FIG. 1). When the needle hub is connected to the needle holder 42, tabs on the hub engage threads 50 formed on the inner wall of a sleeve 52 surrounding the projection 48. The sleeve 52 is an extension of the needle holder body 54, and the needle holder body has a set of longitudinal ribs 56 formed on its exterior. These ribs are uniformly spaced about the circumference of the needle holder, and have tapered distal ends 58. An elastomeric O-ring 60 is provided in a circumferential groove 62 formed on the exterior of the needle holder body 54 at a location spaced proximally from the proximal ends of the ribs. The O-ring cooperates with the inner wall of the extension 44 (FIG. 1) to provide a seal that prevents fluid from leaking from the fluid chamber 24 through a space between the needle holder and the inner wall of the extension.

FIGS. 3 and 4 show a molded resin insert 64, which fits into an opening 66 (FIG. 2) at the proximal end of the needle holder. The insert has a distal flange 68, which snaps into a groove 70 after passing over a ring 72 on the inner wall of opening 66 when the insert is pressed into opening 66. The ring 72 holds the insert in place in the needle holder, restraining the insert against proximal movement relative to the needle holder. The flange 68, groove 70, and ring 72 should be shaped so that the axial pulling force required to release the flange from the groove is substantially greater than the maximum axial pulling force required to release the needle holder from the extension of the syringe barrel.

As shown in FIGS. 3 and 4, the insert 64 is formed with a pair of opposed resilient fingers 74 and 76, which are unitary with the insert and extend forward and inward from the base of the insert. These resilient fingers, are disposed in windows formed in the annular side wall 77 of the insert. When the fingers are in their relaxed condition, as shown in FIGS. 3, 5 and 6, their tips are located radially inward with respect to the adjacent parts of the annular sidewall 77 of the insert The projection 46 on the plunger is provided with an enlarged head 78, which has a rounded forward edge and a flat, radial, proximally facing surface 79 (FIGS. 1, 5 and 6), which forms a sharp corner with the outwardly facing part of the head 78. When the plunger is pushed in the distal direction so that its projection 46 enters the insert 64, the rounded part of the head 78 presses outward on the fingers 76, and the head can move past the tips of the fingers. When the head moves past the tips of the fingers, the resilience of the fingers causes them to snap back into their original positions, as shown in FIG. 6, and their tips can then engage the flat, proximally facing surface 79 of the head 78, thereby securely locking the plunger to the needle holder so that the needle holder can be withdrawn into the barrel by pulling on the plunger.

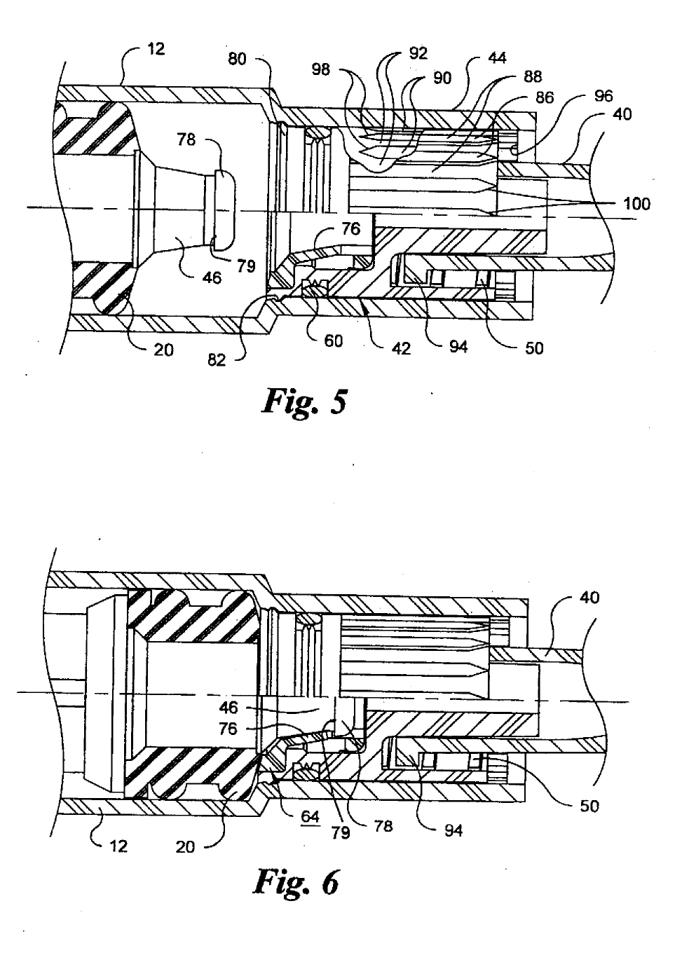

The manner in which the needle holder is engaged and withdrawn is illustrated in FIGS. 5 and 6. In FIG. 5, the needle holder 42 is held in extension 44 of the syringe barrel by engagement of an annular flange 80 formed adjacent the proximal end of the needle holder, with a groove 82 formed on the inner wall of the extension. The flange 80 and the groove 82 have corresponding V-shaped cross-sections. The needle holder 42 and the extension 44 have some degree of resilience, and therefore the flange can be released from the groove by the application of a sufficient proximally directed force to the needle holder. The size and shape of the flange 80 and groove 82 should be such that the force required to release the engagement of the needle holder from the barrel extension is greater than the maximum force normally exerted on the needle in the processes of attaching the needle hub to the needle holder, filling the syringe from a vial, and administering an injection. On the other hand, the size and shape of the flange 80 and groove 82 should be such that the needle holder can be readily released from the barrel extension by a manually applied proximal pulling force exerted on the plunger when the plunger is engaged with the needle holder as shown in FIG. 6. Thus, the mutually engaged retaining elements on the needle holder and in the hollow extension require the magnitude of a proximal pulling force exerted on the plunger to exceed a predetermined magnitude in order to permit proximal movement of the needle holder.

As seen in FIG. 6, the flange 78 on projection 46 of the plunger is located distally in relation to the tips of the resilient fingers, e.g., finger 76, of the insert 64, and the fingers have returned to their relaxed positions, where they can be engaged by the proximal face 79 of flange 78 and remain engaged with the proximal face of the flange when the plunger is pulled in the proximal direction, so that the needle holder, and the needle attached to it, can be withdrawn to a safe position within the interior of the barrel 12.

Resilient fingers could be formed in the needle holder itself, but forming the fingers as unitary parts of the needle holder would result in excessive dead space, excessive structural complexity, or insufficient flexibility of the fingers. The use of a separate insert with resilient fingers affords several advantages. First, the plunger is positively connected to the needle holder by engagement of the resilient fingers with the proximally facing surface of the head of the projection on the plunger. Second, because the insert is formed as a separate element, it is easy to make the fingers highly flexible so that they can be easily bent outward by the camming action of the head of the projection on the plunger. Accordingly, the plunger can be engaged with the needle holder without applying an amount of force significantly exceeding the force required to eject fluid from the syringe. Third, because the insert is a separate element, it is possible to minimize dead space without making the structure excessively complex and difficult to manufacture.

When the needle is withdrawn into the barrel, flange 38 (FIG. 1) comes into locking engagement with insert 30, preventing the plunger from causing the needle to project through the distal end of the barrel extension 44. Notches 84 (FIG. 1) are provided in the plunger to enable the proximal part of the plunger to be broken off easily after the syringe is used and before it is discarded.

As shown in FIG. 5, the needle holder is provided with a set of longitudinal ribs 86, evenly spaced about its circumference by a set of longitudinal grooves 88. The inner wall of the barrel extension 44 is also provided with a set of longitudinal ribs 90, evenly spaced about its circumference by a set of longitudinal grooves 92. The widths of ribs 86 are slightly less than the widths of grooves 92 and the widths of ribs 90 are slightly less than the widths of grooves 88. When the needle holder is installed in the extension 44 of the syringe, the ribs 86 enter grooves 92 and the ribs 90 enter grooves 88. The engagement of the ribs and grooves prevents rotation of the needle holder about the barrel axis when the needle hub is rotated to engaged its tabs, e.g., tab 94, with the threads 50 of the needle holder.

As shown in FIG. 5, the grooves 92 preferably terminate at ends 96, adjacent the distal end of the extension 44. The ends of grooves 92 prevent the needle holder from being pulled out of extension 44. However, because the needle holder cannot be pulled out of extension 44, it cannot be installed through the distal end of the extension. Rather, it must be installed through the proximal end of the syringe. Because of the length of the syringe barrel, installation of the needle holder is difficult, and must be accomplished with the aid of a special, elongated, plunger-like installation tool (not shown). To facilitate installation of the needle holder with the aid of the installation tool, the ribs 86 or the ribs 90, and preferably both sets of ribs, have pointed ends. Thus, the ribs 90 on the interior of the extension having pointed proximal ends 98, and the ribs 86 on the needle holder having pointed distal ends 100. The pointed ends facilitate engagement of the ribs and grooves in the process of installing the needle holder. Preferably, each set of ribs consists of at least sixteen ribs, so that only a small amount of rotation of the needle holder is required to align the ribs with corresponding grooves when installing the needle holder.

In summary, the syringe in accordance with the invention incorporates a simple and reliable mechanism for engaging and withdrawing the needle holder, but prevents undesired movement of the needle holder into the interior of the syringe barrel, and provides for minimization of dead space. The cooperating ribs and grooves reliably prevent rotation of the needle holder during attachment or detachment of the needle. Its needle holder cannot be pulled out of the barrel, but the needle holder is nevertheless easily installed from the proximal end by virtue of the pointed ends of the ribs of one or both sets of ribs.

Various modifications can be made to the syringe described above. For example, some of the advantages of the invention can be realized in a version having a positive latching device that is released upon distal movement of the plunger, instead of relying upon friction or cooperating resilient elements to retain the needle holder. The number and configuration of the ribs and grooves can be changed. For example, each set of ribs can consist of fewer than sixteen ribs; some or all of the ribs can be formed without pointed ends; the ribs can be provided in various different lengths, and, although uniform spacing is desirable, the ribs can be spaced non-uniformly. The hollow extension of the barrel does not need to be narrower than the barrel, and can be simply a part of the barrel itself with no well-defined transition between the barrel and the extension. These and other modifications that may occur to those skilled in the art can be made to the syringe of the invention without departing from the scope of the invention as defined by the following claims.

What is claimed is:

1. A syringe comprising:
a barrel having a cylindrical inner wall, the barrel being elongated along a longitudinal barrel axis and having a proximal end and a distal end, the barrel having an opening at its proximal end, and a hollow extension at its distal end;
a stopper in fluid-tight sliding engagement with the cylindrical inner wall of the barrel;
a plunger connected to the stopper and extending outward from the barrel through the opening at the proximal end of the barrel;
a needle holder fitted in the hollow extension, the needle holder having a passage extending from a proximal end thereof to a distal end thereof;
coupling elements on the needle holder and the plunger engageable with each other when the plunger is moved distally beyond a predetermined limit, for coupling the plunger to the needle holder and thereby enabling the needle holder, and a needle hub and needle connected to the needle holder, to be withdrawn into the interior of the barrel by the exertion of a proximal pulling force on the plunger; and
mutually engaged retaining elements on the needle holder and in the hollow extension, the retaining elements resisting proximal movement of the needle holder but permitting proximal movement of the needle holder upon application of a proximal pulling force on the plunger when the coupling elements on the needle holder and the plunger are engaged;
wherein the coupling element on the needle holder comprises an insert fitted into the passage in the needle holder and restrained against proximal movement relative to the needle holder, the insert having a base at a proximal end thereof and a side wall at least partially surrounding, and defining, an axial passage through the insert, the side wall being interrupted by a plurality of windows, and a resilient finger disposed in each of said windows and extending distally from the base, each finger being unitary with the base of the insert and having a tip remote from the base, the tip of each finger, when the fingers are relaxed, being located at least partly within the axial passage of the insert;
wherein the coupling element on the plunger comprises an axial protrusion having a head with a proximally facing surface, the head being of a size such that the head is capable of passing through the axial passage of the insert but sufficient to engage the fingers of the insert and spread the tips of the fingers of the insert radially outward as the axial protrusion is moved distally; and
wherein, when the plunger is moved distally by a distance such that the proximally facing surface of the head of the axial protrusion passes the tips of the fingers of the insert, the tips of the fingers are engageable with the proximally facing surface of the head of the axial protrusion and the engagement of the proximally facing surface with the tips of the fingers enables the needle holder, and a needle connected to the needle holder, to be withdrawn into the barrel as the plunger is pulled in the proximal direction.

2. A syringe according to claim 1, including a hollow needle fixed to a needle hub, the needle hub being removably engaged with the needle holder to establish a rigid connection of the needle and needle hub to the needle holder and to establish a fluid-conducting connection of the hollow needle to the passage of the needle holder;

3. A syringe according to claim 1, in which the mutually engaged retaining elements on the needle holder and in the hollow extension require the magnitude of a proximal pulling force exerted on the plunger to exceed a predetermined magnitude in order to permit proximal movement of the needle holder.
